(19) United States
(12) Patent Application Publication
(10) Pub. No.: US 2009/0018206 A1
(43) Pub. Date: Jan. 15, 2009

(54) PREVENTION OF HYPOTENSION AND STABILIZATION OF BLOOD PRESSURE IN HEMODIALYSIS PATIENTS

(75) Inventors: Refael Barkan, Rishon Le Zion (IL); Alexander Mirimsky, Rehovot (IL); Ze've Katzir, Kfar Uriah (IL); Victor Ghicavii, Moldova (IL)

Correspondence Address: WINSTON & STRAWN LLP;PATENT DEPARTMENT, 1700 K STREET, N.W., WASHINGTON, DC, 20006, US

(73) Assignee: MEDITOR PHARMACEUTICALS LTD.

(21) Appl. No.: 12/066,041

(22) PCT Filed: Sep. 6, 2006
(86) PCT No.: PCT/IL2006/001044

§ 371 Date: Sep. 9, 2008

Related U.S. Application Data
(60) Provisional application No. 60/713,719, filed on Sep. 6, 2005.
(60) Provisional application No. 60/778,841, filed on Mar. 6, 2006.

(51) Int. Cl. A61K 31/14; A61P 9/02

(52) U.S. Cl. 514/631

(57) ABSTRACT

The present invention relates to the use of S-alkylisothiouronium derivatives, including S-ethylisothiouronium diethylphosphate, for stabilizing blood pressure in hemodialysis patients. The compositions of the invention are effective in preventing hypotension in hemodialysis patients.

### FIELD OF THE INVENTION

The present invention relates to the use of S-alkylisothiouronium derivatives for preventing hypotension in hemodialysis patients. In particular, the present invention relates to methods for the prevention of hypotension and stabilization of blood pressure in hemodialysis patients.

### BACKGROUND OF THE INVENTION

Chronic renal failure (CRF) may result from any major cause of renal dysfunction. The most common cause of end-stage renal disease is diabetic nephropathy, followed by hypertensive nephroangiosclerosis and various primary and secondary glomerulopathies. The functional effects of CRF can be categorized as diminished renal reserve, renal insufficiency (failure), and uremia.

Treatments for CRF include protein restriction, angiotensin-converting enzyme (ACE) inhibitors, possibly angiotensin receptor blockers, and meticulous attention to diet as CRF progresses from moderate to end-stage disease. When conventional therapy is no longer effective, the patient is considered to have end-stage renal disease (ESRD) and long-term dialysis or transplantation is an option. Most physicians agree that uremic symptoms (nausea, vomiting, anorexia, fatigability, diminished sensorium) and signs (pericardial friction rub, refractory pulmonary edema, metabolic acidosis, foot or wrist drop, asterixis) necessitate urgent dialysis.

Dialysis provides a method for supplementing or replacing renal function in ESRD patients. Dialysis is the process of separating elements in a solution by diffusion across a semipermeable membrane (diffusive solute transport) down a concentration gradient. Principally, hemodialysis (directly from the blood) and peritoneal dialysis (indirectly via peritoneal fluid) are utilized.

A dialysis regimen for ESRD should improve the patient's ability to perform activities of daily living, improve comfort, allow the patient to eat a reasonable diet, help maintain a normal blood pressure, and prevent progression of uremic neuropathy. Most ESRD patients require hemodialysis thrice weekly to maintain a state of well-being. Early treatment typically takes three to five hours in adults and three to four hours in children. Blood is removed from the patient via a suitable vascular access and pumped to the membrane unit. The dialysate compartment of the membrane unit is under negative pressure relative to the blood compartment, which permits hydraulic ultrafiltration of excess total body fluid across the membrane. Dialyzed blood is returned to the patient through tubing with an air embolus protector.

The most common complications during hemodialysis are, in descending order of frequency, hypotention (20-30% of dialyses), cramps (5-20%), nausea and vomiting (5-15%), headache (5%), chest pain (2-5%), back pain (2-5%), itching (5%), and fever and chills (<1%).

Hypotension during dialysis is a very common event. This is usually due to a reduced blood volume consequent to fluid removal by ultrafiltration and the patient's inability to physiologically compensate for the reduced blood volume.

Hemodynamic dysregulation in hemodialysis (HD) patients, specifically intradialytic hypotension, which occurs in up to 20% of dialysis sessions, is associated with poor patient outcome (Daugirdas J T. Am J Kidney Dis 2001; 38:S-11). There is evidence that life-threatening conditions such as non-occlusive mesenteric ischemia are associated with frequent intradialytic hypotension and corollary damage to brain and cardiac tissue might also be expected to occur in patients with frequent hypotensive episodes.

Normally, the removal of water and solutes from the blood is compensated by plasma refilling and reduction of venous capacity, a response to reduced transmission of pressure to the veins. During the HD session, a large volume of water and solutes from the blood are removed over a short period of time, overwhelming the normal compensatory mechanisms. In some patients, the blood volume reduction may be accompanied by an inappropriate reduction of sympathetic tone, leading to reduced arteriolar resistance, increases transmission of pressure to the veins and increased venous capacity. This increased venous sequestration of blood reduces cardiac filling, cardiac output and blood pressure.

Current protective maneuvers for intradialytic hypotension include ultrafiltration rate reduction by use of longer dialysis sessions, more frequent dialysis treatments (Sherman R A. Am J Kidney Dis 2001; 38: S18-25), and cool temperature dialysis in which the dialysate has been cooled to approximately 35° C. (Dheenan S et al., Kidney Int 2001; 59: 1175-81). These treatments, however, may represent physiologic pitfalls as well as logistic impossibilities in the busy dialysis center. The goal of a thermal prescription in hemodialysis is to maintain the core (or arterial blood) temperature at its initial level during the dialysis treatment. This goal is not readily achievable without sophisticated thermal balancing techniques for several reasons. First, the predialysis core temperature varies by more than 1° C. among patients and frequently within an individual patient between treatments. Thus the initial dialysate temperature needed to maintain isothermia would vary substantially among patients. Second, ultrafiltration may induce vasoconstriction, which reduces heat loss. This phenomenon necessitates a progressively cooler dialysate to maintain thermal balance (Rosales L M et al., Am J Kidney Dis 2000; 36: 353-61). Third, heat is lost to the environment from the venous line at a rate that is proportional to blood flow. Increased dialysis solution sodium chloride is another optional treatment modality that can maintain blood volume and refilling, but may also increase interdialytic thirst in a fluid-restricted population. Dietary sodium restriction is often met with poor compliance.

Patients at risk for the development of intradialytic hypotension are those with a history of hypovolemia, heart failure, left ventricular hypertrophy, atrial fibrillation, older age (>60 years) or diabetes mellitus (Sherman R A. Semin Dial 2002; 15:141-3).

U.S. Pat. No. 6,271,228 of Grossman et al., discloses a method for stabilizing blood pressure during hemodialysis, which uses a phosphodiesterase inhibitor in the treatment of humans.

WO 98/13036 of Mizrakh et al., discloses the use of S-alkylisothiouronium derivatives, as medicaments for increasing arterial blood pressure or for protecting subjects against hyperoxia. These compounds are suggested for the treatment of acute hypotension, e.g., shock conditions and chronic arterial hypotension or oxygen poisoning. The invention is exemplified by the hypertensive effect of S-ethylisothiouronium diethylphosphate under various conditions. However, WO 98/13036 neither teaches nor suggests the use of S-alkylisothiouronium derivatives for the prevention of hypotension in hemodialysis patients.

WO 02/19961 of Barkan et al., discloses the use of S-alkylisothiouronium derivatives, for the prevention or treatment of headache, including migraine.

Hypotension remains the most prevalent side effect of hemodialysis and although its incidence has diminished with the advent of more advanced dialysis technology, the management treatments described above are not wholly satisfactory. For example, they include interruption of dialysis for a period to allow for blood pressure normalization. Thus, there is a continuing need for an alternative treatment for hypotension consequent to hemodialysis.

### SUMMARY OF THE INVENTION

The present invention provides methods and compositions for preventing hypotension in hemodialysis patients. In particular, the present invention discloses the unexpected finding that the use of S-alkylisothiouronium derivatives before or during hemodialysis prevents hypotension and stabilizes blood pressure.

Thus, according to one aspect, the present invention provides a method for the prevention of hypotension in a subject receiving hemodialysis comprising administering to the subject a therapeutically effective amount of a compound having the general formula I:

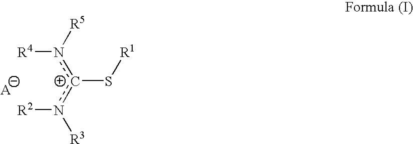

wherein,

R¹ is a linear or branched, saturated or unsaturated alkylene, comprising one to eight carbon atoms, optionally substituted with one or more substituents selected from the group consisting of halogen, primary, secondary, tertiary or quaternary amine, primary, secondary or tertiary alcohol, or interrupted by one or more heteroatom selected from the group consisting of O, N, and S;

R², R³, R⁴ and R⁵ are each independently a hydrogen, hydroxy, an alkylene including linear or branched lower alkyl, linear or branched lower alkenyl, linear or branched lower alkynyl, lower alkoxy, alkoxyalkyl, cycloalkyl, cycloalkylalkyl, lower thioalkoxy, nitro, amino, cyano, sulfonyl, haloalkyl, carboaryloxy, carboalkylaryloxy, alkyl sulfoxide, aryl sulfoxide, alkyl sulfone, aryl sulfone, alkyl sulfate, aryl sulfate, sulfonamide, thioalkyl, optionally substituted by halogen;

A⁻ is a physiologically acceptable anion; and a pharmaceutically acceptable carrier or diluent.

According to one embodiment of the present invention, the physiologically acceptable anion is selected from the group consisting of an anion derived from a phosphorus containing acid, a phosphorus acid ester and a phosphorus acid amide, preferably the anion is derived from a mono or di-alkyl ester of a phosphate or phosphite.

In other embodiments the physiologically acceptable anion is selected from the group consisting of an anion derived from a phosphorus containing acid, a phosphorous acid ester, a phosphorous acid amide, acetate, adipate, alginate, citrate, aspartate, benzoate, benzenesulfonate, bitartarate, bisulfate, butyrate, camphorate, camphorsulfonate, digluconate, glycerophosphate, hemisulfate, heptanoate, hexanoate, fumarate, 2-hydroxyethanesulfonate, isothionate, lactate, maleate, methanesulfonate, nicotinate, 2-naphthalenesulfonate, oxalate, palmoate, pectinate, 3-phenylpropionate, pivalate, propionate, succinate, tartrate, thiocyanate, glutamate, bicarbonate, p-toluenesulfonate, chloride, bromide, iodide and undecanoate.

In yet other embodiments each of R², R³, R⁴ and R⁵ is hydrogen. In some embodiments R¹ is a linear or branched alkyl.

Accordingly, in one embodiment the S-alkylisothiouronium derivative is a compound of formula (II):

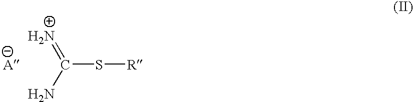

wherein

R″ is a straight or branched alkyl, optionally substituted by halogen; and

A″(−) is an anion derived from a phosphorous containing acid.

The present invention further provides use of a compound having general formula (I) or (II) for the manufacture of a medicament for use in the prevention of hypotension in hemodialysis patients.

According to some embodiments the compound is selected from the group consisting of:

S-methylisothiouronium methylphosphite; S-methylisothiouronium dimethylphosphate; S-ethylisothiouronium metaphosphate; S-ethylisothiouronium ethylphosphite; S-ethylisothiouronium diethylphosphate; b S-propylisothiouronium propylphosphite; S-isopropylisothiouronium metaphosphate; S-isopropylisothiouronium isopropylphosphite; S-butylisothiouronium dibutylphosphate; and S-isobutyl-isothiouronium isobutylphosphite.

In certain embodiments the compound is S-ethylisothiouronium diethylphosphate.

According to still further features in the described preferred embodiments the anti-hypotension medicament is formulated for parenteral modes of administration. Among the parenteral routes of administration particularly preferred formulations are suitable for injection, or infusion administration. Another preferred route of administration is oral administration.

According to one embodiment the anti-hypotension medicament is administered before the hemodialysis.

According to another embodiment, the anti-hypotension medicament is administered during the hemodialysis.

According to some embodiments the therapeutically effective amount suitable for injection, or infusion administration ranges between 0.1 and 5 mg/kg body weight. According to other embodiments said therapeutically effective amount ranges between 0.1 and 2.4 mg/kg body weight. According to some embodiments said therapeutically effective amount ranges between 0.3 and 2.4 mg/kg body weight. According to other embodiments said therapeutically effective amount ranges between 0.5 and 1.8 mg/kg body weight. According to other embodiments said therapeutically effective amount ranges between 0.5 and 1.2 mg/kg body weight.

According to other embodiments the therapeutically effective amount suitable for oral administration ranges between 0.1 and 2.4 mg/kg body weight.

These and other embodiments of the present invention will become apparent in conjunction with the figures, description and claims that follow.

### BRIEF DESCRIPTION OF THE FIGURES

FIGS. 1A-1D show the effect of an injectable formulation of S-ethylisothiouronium diethylphosphate (MTR107) on blood pressure during hemodialysis.

FIG. 2 shows the pharmacokinetic model applied for the analysis of MTR107 concentration vs. time data in hemodialysis patients.

FIG. 3 shows a linear plot of MTR107 concentration vs. time curves following intravenous (IV) administration to hemodialysis patients.

FIG. 4 shows a linear plot of average observed MTR107 concentrations (data points) and predicted concentrations according to the compartment model (solid lines) values following IV administration of MTR107 to humans.

FIG. 5 shows a linear plot of predicted MTR107 concentrations in hemodialysis patients for different doses of the drug.

FIGS. 6A-6E show linear plots of predicted MTR107 concentrations in hemodialysis patients for different doses of the drug.

FIG. 7 shows a semi-logarithmic plot of MTR107 concentrations following the administration of the 1^{st} and the 6^{th} doses of the drug (0.3 mg/kg) to the hemodialysis patients (the administration time of each dose was set to 0).

FIG. 8 shows a semi-logarithmic plot of MTR107 concentrations following the administration of the 1^{st} and the 6^{th} doses of the drug (2.4 mg/kg) to the hemodialysis patients (the administration time of each dose was set to 0).

FIG. 9 shows a linear plot of predicted MTR107 concentrations in hemodialysis patients for different doses of the drug, assuming that drug body clearance of the patients are negligible (i.e., k₁₀=0).

FIGS. 10A-10E show linear plots of predicted MTR107 concentrations in hemodialysis patients for different doses of the drug, assuming that drug body clearance of the patients is negligible (i.e., k₁₀=0).

FIG. 11 shows a linear plot of predicted MTR107 concentrations in hemodialysis patients for sequentially decreasing doses of the drug, assuming that drug body clearance of the patients is negligible (i.e., k₁₀=0).

FIGS. 12A-12E show linear plots of predicted MTR107 concentrations in hemodialysis patients for sequentially decreasing doses of the drug, assuming that drug body clearance of the patients is negligible (i.e., k₁₀=0).

FIG. 13 shows a semi-logarithmic plot of MTR107 concentrations following the administration of the 1^{st} and the 6^{th} doses of the drug to the hemodialysis patients, for sequentially decreasing doses scenario starting with 2.4 mg/kg, assuming that drug body clearance of the patients is negligible (the administration time of each dose was set to 0).

### DETAILED DESCRIPTION OF THE INVENTION

The present invention relates to the use of S-alkylisothiouronium derivatives, including, but not limited to, S-ethylisothiouronium diethylphosphate, for the prevention of hypotension.

The present invention for the first time discloses the finding that the use of S-alkylisothiouronium derivatives before or during hemodialysis prevents hypotension and stabilizes blood pressure.

### Definitions

As used herein, the term “hypotension” means a hemodynamic condition characterized by reduced blood pressure, which persists despite the maintenance of normal blood volume (normovolemia). Generally, a patient is suffering from hypotension when the mean arterial pressure is less than 90 mm Hg for at least one hour despite adequate ventricular filling pressures (pulmonary artery wedge pressure (PAWP) of at least 12 mm Hg) or despite a sufficient central venous pressure (CVP) of at least 8 mm Hg. Other indicators of hypotension are the failure of the hypotensive state to respond to aggressive initial fluid therapy (such as the administration of 500 ml of isotonic crystalloid, 25 gm or albumin, or 200 ml of other colloids (e.g. hydroxyethyl starch) or the need for pressor doses of dopamine (>5 g/kg/min), norepinephrine or other pressor agents to maintain a systolic blood pressure of 90 mm Hg.

The term “intradialytic hypotension (IDH)” is defined herein in patients with pre-dialysis blood pressure ≦120 mmHg as a decrease in systolic blood pressure (SBP) or mean arterial pressure (MAP) from the pre-dialylitic baseline of both values. In some instances the decrease is of about 20%.

As used herein, the term “predisposition for intradialytic hypotension” refers to a patient who experiences recurrent episodes of intradialytic hypotension at least thrice per month for the last six months despite standard adjustments in dry weight and changes in anti-hypotensive medications.

As used herein, the term “subject” refers to a mammal, including both human and other mammals. The methods of the present invention are preferably applied to human subjects.

As used herein the term “therapeutically effective amount” or “therapeutically efficient” as to a drug dosage, refer to dosage that provides the specific pharmacological response for which the drug is administered in a significant number of subjects in need of such treatment. The “therapeutically effective amount” may vary according, for example, the physical condition of the patient, the age of the patient and the severity of the hypotension.

The term “MTR107” as used herein refers to the injectable formulation of S-ethylisothiouronium diethylphosphate.

The term “MTR106” as used herein refers to the oral formulation of S-ethylisothiouronium diethylphosphate.

The term “about” as used herein refers to +/−10%.

As used herein, the term “alkylene” refers to a saturated or unsaturated hydrocarbon chain including straight chain or branched chain alkyl, alkenyl or alkynyl.

As used herein, the term “alkyl” refers to a saturated hydrocarbon chain containing 1 to 30, preferably 1 to 6 carbon atoms, such as, but not limited to, methyl, ethyl, n-propyl, isopropyl, n-butyl, isobutyl, tert-butyl, n-pentyl, n-hexyl, and the like. As used herein the term alkyl also reads on haloalkyls, which contain halogen atoms. Alkyl also includes heteroalkyl with heteroatoms of sulfur, oxygen and nitrogen.

“Alkenyl” and “alkynyl” are used to mean straight or branched chain hydrocarbon groups having from 2 to 12 carbons and unsaturated by a double or triple bond respectively, such as vinyl, allyl, propargyl, 1-methylvinyl, but-1-enyl, but-2-enyl, but-2-ynyl, 1 methylbut-2-enyl, pent-1-enyl, pent-3-enyl, 3-methylbut-1-ynyl, 1,1-dimethylallyl, hex-2-enyl and 1-methyl-1-ethylallyl.

The term “cycloalkyl” is used herein to mean cyclic radicals, including but not limited to, cyclopropyl, cyclopentyl, cyclohexyl, and the like.

The term “cycloalkylalkyl” as used herein refers to a cycloalkyl group appended to a lower alkyl radical, including, but not limited to cyclohexylmethyl. The “alkoxyalkyl” mentioned for R substitutes is preferably a group containing a total of 1-22 carbon atoms. As example, methoxyethyl, methoxypropyl, methoxybutyl, ethoxyethyl, ethoxypropyl, ethoxybutyl, n-propoxyethyl, and iso-propoxyethyl, can be mentioned.

The term “alkoxy” as used herein refers to an alkyl group attached to the parent molecular group through an oxygen atom.

The term “alkoxyalkoxy” as used herein refers to an alkoxy group attached to the parent molecular group through an alkoxy group.

The term “halo” or “halogen” as used herein refers to I, Br, Cl or F.

The term “carboxy” as used herein refers to the radical —COOH. The term “ester” refers to —COOR; and the term “amide” refers to —CONH₂ or —CONHR or —CONR₂. The term “cyano” as used herein refers to the radical —CN.

As used herein a “pharmaceutical composition” refers to a preparation of one or more of the compounds described herein, or physiologically acceptable salts or prodrugs thereof, with other chemical components such as physiologically suitable carriers and excipients. The purpose of a pharmaceutical composition is to facilitate administration of a compound to an organism.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

### Preferred Embodiments of the Present Invention

Without excluding other options, which are listed below, S-ethylisothiouronium diethylphosphate is at present the preferred compound for preventing hypotension in hemodialysis patients. S-ethylisothiouronium diethylphosphate is now shown to be an effective agent for preventing hypotension in hemodialysis patients.

According to one aspect of the present invention there is provided an anti-hypotension medicament for hemodialysis patients comprising, as an active ingredient, a compound having the general formula (I):

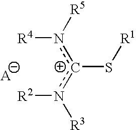

wherein,
R¹ is a linear or branched saturated or unsaturated alkylene, comprising one to eight carbon atoms optionally substituted with one or more substituent selected from the group consisting of halogen, primary, secondary, tertiary or quaternary amine, primary, secondary or tertiary alcohol, or interrupted by one or more heteroatom selected from the group consisting of O, N, and S;
R², R³, R⁴ and R⁵ are each independently a hydrogen, hydroxy, linear or branched lower alkyl, linear or branched lower alkenyl, linear or branched lower alkynyl, lower alkoxy, alkoxyalkyl, cycloalkyl, cycloalkylalkyl, lower thioalkoxy, nitro, amino, cyano, sulfonyl, haloalkyl, carboaryloxy, carboalkylaryloxy, alkyl sulfoxide, aryl sulfoxide, alkyl sulfone, aryl sulfone, alkyl sulfate, aryl sulfate, sulfonamide, thioalkyl, optionally substituted by halogen;
A⁻ is a physiologically acceptable anion;
and a pharmaceutically acceptable carrier or diluent.

Preferably, the physiologically acceptable anion is derived, without limitation, from a phosphorus containing acid, the group consisting of an anion derived from a phosphorus containing acid, acetate, adipate, alginate, citrate, aspartate, benzoate, benzenesulfonate, bitartarate, bisulfate, butyrate, camphorate, camphorsulfonate, digluconate, glycerophosphate, hemisulfate, heptanoate, hexanoate, fumarate, hydrochloride, 2-hydroxyethanesulfonate, isothionate, lactate, maleate, methanesulfonate, nicotinate, 2-naphthalenesulfonate, oxalate, palmoate, pectinate, 3-phenylpropionate, pivalate, propionate, succinate, tartrate, thiocyanate, phosphate, glutamate, bicarbonate, p-toluenesulfonate, chloride, bromide, iodide and undecanoate.

According to currently preferred embodiments of the invention described below, the physiologically acceptable anion is an anion derived from a phosphorus containing acid, more preferably the group consisting of an anion derived from a phosphorus acid ester or amide, most preferably the anion is derived from a mono or di-alkyl ester of a phosphorous containing acid.

Other examples of S-alkylisothiouronium derivatives which can be used to prevent and/or treat hypotension in hemodialysis patients, according to the present invention include, but are not limited to S-methylisothiouronium methylphosphite; S-methylisothiouronium dimethylphosphate; S-ethylisothiouronium metaphosphate; S-ethylisothiouronium ethylphosphite; S-ethylisothiouronium diethylphosphate; S-propylisothiouronium propylphosphite; S-isopropylisothiouronium metaphosphate; S-isopropylisothiouronium isopropylphosphite; S-butylisothiouronium dibutylphosphate; and S-isobutylisothiouronium isobutylphosphite.

These compounds are known to be safe for human use, as it is well known in the art that phosphorus containing derivatives of S-alkylisothiouronium have a low toxicity and their LD₅₀ (lethal dose 50%) is in the range of 100-1000 mg/kg, which is far above the therapeutic doses of these compounds.

The toxicological studies indicated that the compounds of the invention are not toxic when administered as either a single or repeated dose. For example, the LD₅₀ for MTR107 is up to 400 mg/kg in rats, values 300-400 fold higher than the therapeutically recommended dose of 0.1-2.4 mg/kg.

According some embodiments the anti-hypotension medicament is administered before the hemodialysis procedure. According to other embodiments, the anti-hypotension medicament is administered during the hemodialysis procedure.

According to some embodiments the therapeutically effective amount suitable for oral administration ranges between 0.1 and 2.4 mg/kg body weight.

In another aspect of the present invention there is provided a method for preventing hypotension in hemodialysis patients. The method according to this aspect of the present invention is effected by administering to a subject a therapeutically effective amount of a compound having the general formula (I):

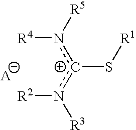

wherein
R¹ is a linear or branched saturated or unsaturated alkylene, comprising one to eight carbon atoms optionally substituted with one or more substituent selected from the group consisting of halogen, primary, secondary, tertiary or quaternary amine, primary, secondary or tertiary alcohol, or interrupted by one or more heteroatom selected from the group consisting of O, N, and S;
R², R³, R⁴ and R⁵ are each independently a hydrogen, hydroxy, linear or branched lower alkyl, linear or branched lower alkenyl, linear or branched lower alkynyl, lower alkoxy, alkoxyalkyl, cycloalkyl, cycloalkylalkyl, lower thioalkoxy, nitro, amino, cyano, sulfonyl, haloalkyl, carboaryloxy, carboalkylaryloxy, alkyl sulfoxide, aryl sulfoxide, alkyl sulfone, aryl sulfone, alkyl sulfate, aryl sulfate, sulfonamide, thioalkyl, optionally substituted by halogen;
A⁻ is a physiologically acceptable anion;
and a pharmaceutically acceptable carrier or diluent.

### Pharmaceutical Composition of the Present Invention

A compound according to the present invention can be administered to a treated subject per se, or in a pharmaceutical composition where it is mixed with suitable carriers or excipients.

Pharmaceutical compositions may also include one or more additional active ingredients, such as, but not limited to, conventional anti-hypotension agents.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

For injection, the compounds of the invention may be formulated in aqueous solutions, carrier or diluent, preferably in physiologically compatible buffers such as Hank's solution, Ringer's solution, phosphate buffer or physiological saline buffer.

For transmucosal administration, penetrants appropriate to the barrier to be permeated are used in the formulation. Such penetrants for example DMSO, or polyethylene glycol are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the compounds for use according to the present invention are conveniently delivered in the form of an aerosol spray presentation from a pressurized pack or a nebulizer with the use of a suitable propellant, e.g., dichlorodifluoromethane, trichlorofluoromethane, dichlorotetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Pharmaceutical compositions for parenteral administration include aqueous solutions of the active preparation in water-soluble form. Additionally, suspensions of the active compounds may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the compounds, to allow for the preparation of highly concentrated solutions.

Alternatively, the active ingredient may be in powder form for constitution with a suitable vehicle, e.g., sterile, pyrogen-free water, before use.

The compounds of the present invention may also be formulated in rectal compositions such as suppositories or retention enemas, using, e.g., conventional suppository bases such as cocoa butter or other glycerides.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions suitable for use in context of the present invention include compositions wherein the active ingredients are contained in an amount effective to achieve the intended purpose. More specifically, a therapeutically effective amount means an amount of a compound effective to prevent, alleviate or ameliorate hypotension in the subject being treated.

Determination of a therapeutically effective amount is well within the capability of those skilled in the art, especially in light of the detailed disclosure provided herein.

The exact formulation, route of administration and dosage can be chosen by the individual physician in view of the patient's condition. (See e.g., Fingl, et al., 1975, in “The Pharmacological Basis of Therapeutics”, Ch. 1 p. 1).

The amount of a composition to be administered will, of course, be dependent on the subject being treated, the severity of the hypotension, the manner of administration, the judgment of the prescribing physician, etc. For example, doses up to 2.4 mg/kg of MTR107 would be well tolerated in healthy volunteers and represents a therapeutic alternative for the treatment of hypotension in hemodialysis patients.

A pharmaceutical composition containing S-alkylisothiouronium may be used either before or during the hemodialysis procedure. According to one embodiment the pharmaceutical composition of the invention is administered before the initiation of hemodialysis and it is especially preferred that the pharmaceutical composition of the invention is administered by intravenous injection or by oral administration before the hemodialysis procedure.

According to another embodiment of the invention, hemodialysis occurs with a dialyzer or dialysis tubing that is internally rinsed with a solution of S-alkylisothiouronium. According to a further embodiment of the invention, the administration of the amount of the S-alkylisothiouronium derivative is titrated to the blood pressure of the hemodialysis patient.

Single or multiple administrations of the compositions of the invention can be carried out. Furthermore, constant, variable, decreasing, or escalating doses may be employed.

Microparticles and nanoparticles can be used for sustained drug release in the present invention. Microparticles and nanoparticles employ small biodegradable spheres which act as depots for delivery. The major advantage of polymer microspheres is that they are extremely safe and have been approved by the Food and Drug Administration in the US for use in human medicine as suitable sutures and for use as a biodegradable drug delivery system (Langer, 1990, Science, 249 (4976):1527-33). The rates of polymer hydrolysis are very well characterized, which in turn allows for the manufacture of microparticles with sustained drug release over prolonged periods of time.

Administration of microparticles elicits long-lasting effect, especially if they incorporate prolonged release characteristics. The rate of release can be modulated by the mixture of polymers and their relative molecular weights, which will hydrolyze over varying periods of time.

Having now generally described the invention, the same will be more readily understood through reference to the following examples, which are provided by way of illustration and are not intended to be limiting of the present invention.

### EXAMPLES

### Example 1

### Formulations and Doses of MTR 107 and MTR 106

Based on animal toxicological studies and on the accumulated data on human patients, MTR107 was approved for a Phase I clinical trial. In this study, the pharmacokinetic profiles as well as safety of constant or escalating doses (0.3-2.4mg/kg) of MTR107 were assessed in 12 healthy male subjects. The results of the Phase I study indicated that MTR107 was well tolerated in doses up to 1.2 mg/kg with no recorded adverse events. Three out of 12 subjects exhibited somnolence as well as transient electrocardiographic alterations during treatment with 2.4 mg/kg (the highest dose). One involved bradycardia, the second involved AV block, and the third was characterized as the occurrence of extrasystoles. Review of pre-treatment (screening) and post study ECGs, as well as 24 hours ambulatory ECGs in these patients, revealed findings that paralleled the on-treatment observations; therefore, the relation of these adverse events to treatment was rated as “unclear” or “possible”.

An example of an injectable formulation is presented in Table 1.

TABLE 1
Exemplary injectable formulation of the present invention (MTR107)

| | Quantity | | |
|---|---|---|---|
| Material | per ml | | Function |
| S-ethylisothiouronium | 100 | mg | Active ingredient |
| diethylphosphate | | | |
| Monosodium Phosphate | 1.59 | mg | Excipient, pH 5.0-6.0 |
| | | | buffer component |
| Disodium Phosphate | 0.33 | mg | Excipient, pH 5.0-6.0 |
| 7H₂O | | | buffer component |

| | | |
|---|---|---|
| Water for Injection | To make up | Excipient, Solvent |

| | | |
|---|---|---|
| (WFI) | 1.00 | ml |

An example of an oral formulation is presented in Table 2.

TABLE 2
Exemplary oral formulation of the present invention (MTR 106)

| Material | Quantity per tablet | |
|---|---|---|
| S-ethylisothiouronium | 50 | mg |
| diethylphosphate | | |
| Lactose | 101 | mg |
| Colloidal Silicone Dioxide | 1.0 | mg |
| Microcrystalline cellulose | 40 | mg |
| Crospovidone (PVP) | 4.0 | mg |
| Stearic acid | 4.0 | mg |

| | |
|---|---|
| Coating materials | Up to 5% of the weight of the |
| | compressed tablet core |

### Example 2

### MTR107 in Endstage Renal Disease (ESRD) Patients During Hemodialysis

### I. Study Objectives

The purpose of the initial exploratory protocol was to analyze the efficacy of MTR107 in a first single dose 0.9 mg/kg administered as slow intravenous (IV) injection (10 ml diluted solution over 3 minutes). If no adequate blood pressure response was observed in first administration, a second dose of 1.2 mg/kg was administered, after a washout period of 72 hours. The study was conducted in ESRD patients with a predisposition for recurrent hypotensive episodes during hemodialysis sessions. The short-term safety and tolerability profile of MTR107 administered during hemodialysis was also evaluated and recorded in this set of patients. Plasma levels of MTR107 in ESRD patients on hemodialysis were measured, and the pharmacokinetic parameters were calculated. Hemodynamic effects at baseline and during hemodialysis were recorded and monitored. Measured hemodynamic parameters were: systolic (SBP), diastolic (DBP), mean arterial blood pressure (MAP), Heart rate (HR), respiration rhythm, and oxygen saturation.

The number of intradialytic hypotensive episodes at baseline was recorded. The pre-dialysis and post-dialysis patient's weight, volume of fluids administered during dialysis, volume of fluids removed at end of dialysis, and change in scheduled length of dialysis session were recorded.

The changes in clinical manifestations commonly associated with intradialytic hypotension at baseline and during treatment with MTR107 were recorded. Common clinical manifestations associated with intradialytic hypotension included loss of consciousness, patient-reported nausea and vomiting, muscle cramps and sweating were recorded.

Primary safety parameters included: systolic and diastolic blood pressure, mean arterial blood pressure, heart rate and oxygen saturation, were measured at baseline, every 5 minutes for the first 30 minutes, thereafter every 10 minutes up to two hours, and every thirty minutes until the end of dialysis. After dialysis end these parameters were recorded at 1-hour intervals for 8 hours post dialysis. All hemodynamic readings were obtained directly from the monitor in triplicates. A printout of the hemodynamic parameters were printed, and used to analyze extreme values throughout the hemodialysis session.

### II. Study Protocol

The study was performed as an open label study in hemodialysis patients with a history of several hypotension episodes during hemodialysis, using baseline characteristics of the same patients as control values. The patients received 0.9 mg/kg MTR107. The hemodialysis was started 10 min before the drug administration and was terminated 240 min after the drug administration. The stock solution of MTR107 was drawn using a 1 ml sterile disposable syringe, and was diluted with saline solution in a total volume of 10 ml. The total volume was injected slowly over 3 minutes to the port entering the body (and after the dialyzer). The blood samples were drawn from the port leaving the body before entering the dialyzer at 0, 5, 10, 15, 20, 30, 45, 60, 90, 120, 150, 180, 240, 360, 480, 600 and 720 min after the drug administration. Plasma was separated and frozen, and MTR107 concentrations were determined.

### III. Selection of Study Population

The records of all patients receiving maintenance hemodialysis as their means of renal replacement therapy at the Department of Hemodialysis at the Republican Hospital, Kishinev, Moldova was reviewed by the medical staff to identify all patients with a history of dialysis hypotension (>3 dialysis hypotensive event per month for the last six months prior to baseline). The medical records of all patients so identified were reviewed to determine eligibility according to the following inclusion/exclusion criteria.

Inclusion criteria: Patients aged 20-75 years inclusive were eligible for study participation if they experienced frequent bouts of hypotension (≧3 dialysis hypotensive events per month for the last six months prior to baseline) during dialysis despite standard adjustments and changes in anti-hypotensive medicine that would be instituted initially to treat the problem.

Exclusion criteria: Patients were excluded if they had uncontrolled hypertension >140/90 mm Hg, unstable angina, variable weight gains (an increase of more than 10 kg measured in between 2 consecutive dialysis), mental retardation, pregnancy, and malignancy or other concomitant serious diseases.

### IV. The Effect of MTR107 on Blood Pressure During Hemodialysis

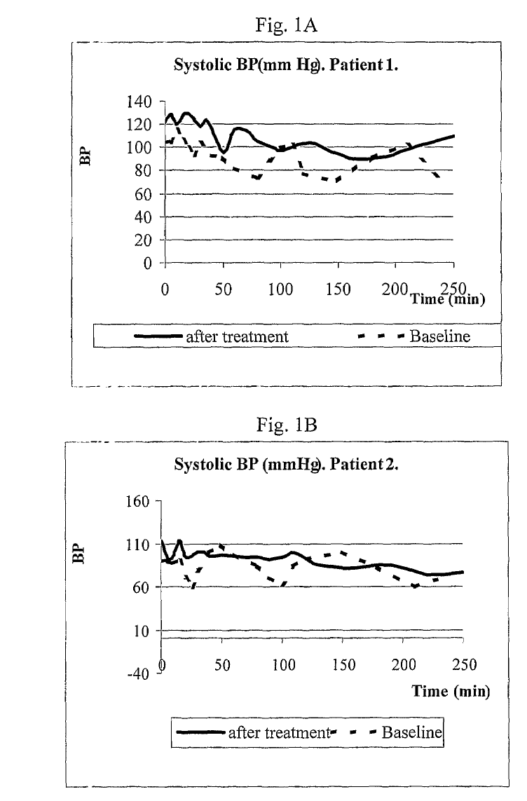

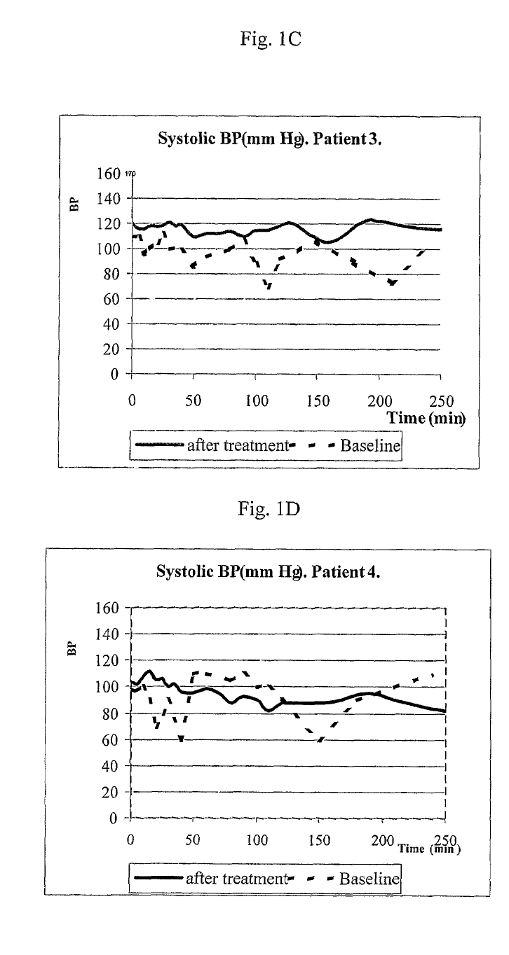

As shown in FIGS. 1A-1D, MTR107 (0.9 mg/kg) normalized blood pressure for approximately two hours during the hemodialysis session, requiring no additional medical intervention. For comparison, baseline (treatment without the drug) hemodynamic data were collected during two dialysis sessions in the same patients. These baseline data demonstrated that each of the hypotension predisposed patients required at least 3 to 4 medical interventions during the session to normalize the blood pressure. In contrast, in the presence of MTR107, the patients' blood pressure was significantly more stable during the hemodialysis.

### V. The Pharmacokinetic Analysis

Pharmacokinetic parameters of MTR107 administered as a single intravenous injection of 0.9 mg/kg were evaluated. The pharmacokinetic parameters that were calculated included: total clearance (CL), volume of distribution at steady state (Vss), volume of distribution (V), half life (t_{1/2}), mean residence time (MRT), and hemodialysis clearance (CLr). The time points for the collection of blood samples (6 ml) were: 0 (before administration), 5 min, 10 min, 15, min, 20 min, 30 min, 45 min, 60 min, 90 min, 120 min, 150 min and 180 min during hemodialysis session and thereafter, every hour for the next 8 hours.

Pharmacokinetic characteristics were estimated from the plasma concentration versus time courses as follows:
The area under the concentration time curve from the time point of drug administration (used as t1=0, C1=0) up to time point of the last quantifiable concentration (AUCₗₐₛₜ) was determined with the linear trapezoidal rule according to the following formula:

$${AUC}_{last} = 0.5 \times {\sum }_{i = 1}^{n - 1} ( {C}_{i} + {C}_{i + 1} ) \times ( {t}_{i + 1} - {t}_{i } )$$

where
i=sampling number
n=total number of quantifiable plasma samples including the time of drug administration (used with t1=0, C1=0)
ti=sampling time corresponding to sample no. i
Ci=concentration at sampling time I

$$Hemodialysis clearance ( {CL}_{HD} ) = Q \frac{( A - V )}{A}$$

### Where

Q is the dialyzer blood flow
A is the drug concentration in blood entering the dialyzer
V is the drug concentration leaving the dialyzer
The area under the concentration time curve from the time point of drug administration (used as t1=0, C1=0) to infinity (AUC_{inf}) was determined with the following formula:

$$AUC inf = AUClast + \frac{Cn}{λ z}$$

The volume of distribution was calculated as:

$${V}_{z} = \frac{CL}{{λ}_{z}}$$

where CL was the Total Clearance and λz was the terminal elimination rate constant.
The Total Clearance was calculated as:

CL=Dose/AUC_{inf}
The peripheral distribution phase was observed from the plasma concentration/time curves.
The terminal elimination rate constant (λz=lambda z) was estimated by linear least squares regression with the logarithmical concentration data of the terminal part of the concentration time curve.
The terminal half-life was determined with the formula:

$$t 1 / 2 = \frac{ln ( 2 )}{λ z}$$

Dose linearity: To check whether there was a dose-linearity, the mean AUCₗₐₛₜ and AUC_{inf} for the different dose groups were depicted graphically.

The noncompartmental analysis was performed applying Nelder-Mead algorithm, with uniform weighting.

The compartmental analysis was performed applying Nelder-Mead algorithm; the weighting applied for the individual subjects was: H2, H4—uniform weighting, H1, H3—1/Y, H5—1/Y².

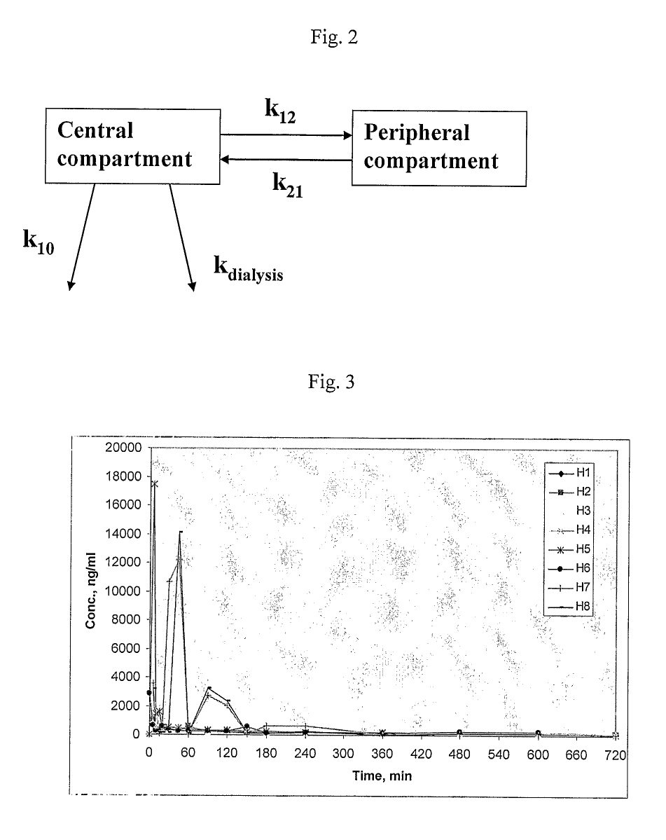

The compartmental analysis applied 2-compartment pharmacokinetic model with two elimination pathways from the central compartment due to body clearance and dialysis as depicted in FIG. 2.

All the drug transfer mechanisms were assumed to follow first order kinetics. The rate constants were: k₁₂—drug transfer from the central to the peripheral compartment, k₂₁—drug transfer from the peripheral to the central compartment, k₁₀—drug elimination from the central compartment due to body clearance processes, k_{dialysis}—drug elimination from the central compartment due to hemodialysis process. The k_{dialysis} was set to 0 at the time periods when the hemodialysis was not performed.

Compartmental analysis was not applied to the concentration vs. time data of subjects 6, 7, and 8 because the curve shapes were unsuitable to compartmental modeling.

### VI. The Pharmacokinetic Analysis Results

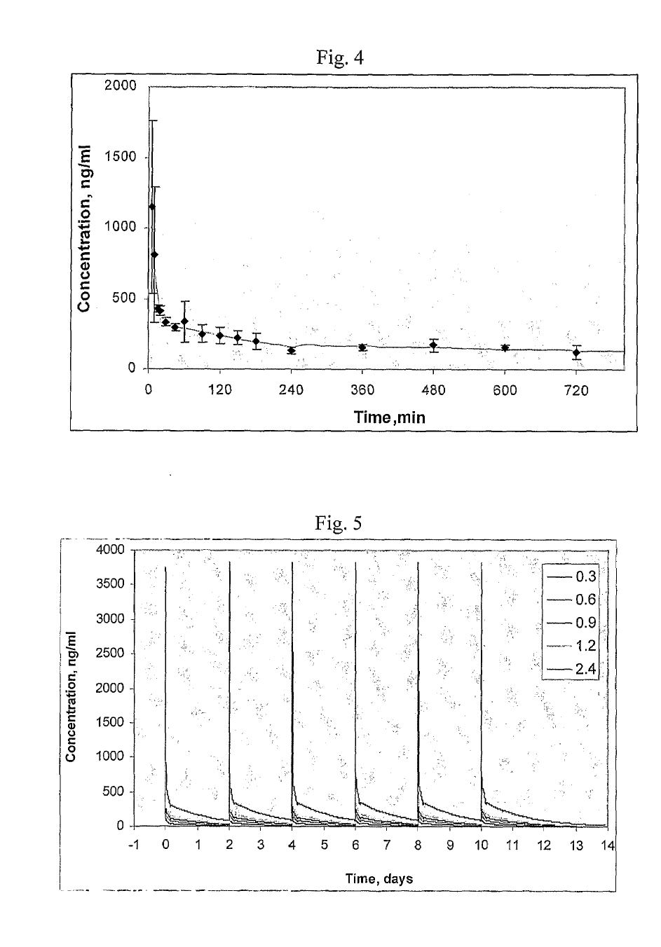

The results of non-compartmental and compartmental pharmacokinetic analysis of the concentration vs. time data are presented in Tables 3-5 and FIGS. 3-4.

TABLE 3
Concentration of MTR107 in human plasma samples following IV administration, ng/ml

| Time | | H1 | H2 | H3 | H4 | H5 | H6 | H7 | H8 |
|---|---|---|---|---|---|---|---|---|---|
| 0 | min | BQL | BQL | 29* | BQL* | 50000# | 2876* | BQL | BQL |
| 5 | min | 685 | 1021 | 850 | 2038 | 17447# | 629 | 3553 | 3162 |
| 10 | min | 484 | 555 | 684 | 1522 | 1442 | 259 | 417 | 278 |
| 15 | min | 444 | 401 | 428 | 446 | 1559 | 291 | 84# | 106 |
| 20 | min | 428 | 379 | 174 | 441 | 536 | 540 | 221 | 109 |
| | | | | 24* | | | | | |
| 30 | min | 354 | 296 | 325 | 359 | 478 | 381 | 10662* | 111 |
| 45 | min | 288 | 271 | 310 | 320 | 457 | 235 | 12205* | 14116* |
| 60 | min | 270 | 549# | 265 | 260 | 397 | 261 | 53 | 36# |
| 90 | min | 236 | 210 | 218 | 342 | 344 | 227 | 2744* | 3236* |
| 120 | min | 223 | 177 | 250 | 314 | 316 | 233 | 2050* | 2365* |
| 150 | min | 208 | 158 | 240 | 280 | 275 | 554# | 82 | 135 |
| 180 | min | 207 | 129 | 192 | 264 | 235 | 156 | 661 | 103 |
| 240 | min | 164 | 126 | 117# | no | 205 | 172 | 620 | 231 |
| | | | | | result# | | | | |
| 6 | hrs | 179 | 155 | 156# | 122# | 192 | 151 | 43 | 20 |
| 8 | hrs | 130 | 146 | 167 | 235 | BQL | 233# | 142 | 138 |
| 10 | hrs | 136 | 166 | 149 | 172 | BQL | 235# | 141 | 139 |
| 12 | hrs | 139 | 153 | 145 | 47 | BQL | BQL | 48 | BQL |

#analysis was repeated due to pharmacokinetic reasons
*analysis was repeated due to analytical reasons
The quantitation limit (QL) was approximately 20 ng/Ml

TABLE 4
Individual and average results of noncompartmental analysis of MTR107 concentration
vs. time data following IV administration to hemodialysis patients

| | | AUC | AUC/D | AUMC | AUMC | | |
|---|---|---|---|---|---|---|---|
| | AUC | % Extrap | min*kg*ng/ | min*min* | % Extrap | MRT | CL |
| Subject | min*ng/ml | % | ml/ng | ng/ml | % | min | ml/min/kg |
| H1 | 285119 | 53.73 | 0.317 | 3.186E+08 | 87.62 | 1116 | 3.16 |
| H2 | 312215 | 57.86 | 0.347 | 3.839E+08 | 89.44 | 1228 | 2.88 |
| H3 | 312062 | 57.30 | 0.347 | 3.901E+08 | 89.53 | 1248 | 2.88 |
| H4 | 184917 | 11.18 | 0.205 | 6.776E+07 | 35.41 | 365 | 4.87 |
| H5 | 265555 | 25.96 | 0.295 | 6.508E+07 | 76.17 | 244 | 3.39 |
| H6 | 708721 | 79.63 | 0.787 | 1.732E+09 | 97.80 | 2442 | 1.27 |
| H7 | 627732 | 2.30 | 0.697 | 8.336E+07 | 17.68 | 131 | 1.43 |
| H8 | 504864 | 9.42 | 0.561 | 9.206E+07 | 48.69 | 181 | 1.78 |
| Average | 400148 | 37.17 | 0.445 | 3.916E+08 | 67.79 | 869 | 2.71 |
| % CV | 47.3 | 76.7 | 47.3 | 143.1 | 44.0 | 91.9 | 44.0 |

| | β | T½ β | Vss | Vβ | Cmax | Cmax/D | Tmax |
|---|---|---|---|---|---|---|---|
| Subject | 1/min | min | ml/kg | ml/kg | ng/ml | ng/ml/mg | min |
| H1 | 0.00091 | 764 | 3522 | 3479 | 685 | 761 | 5 |
| H2 | 0.00085 | 818 | 3540 | 3404 | 1021 | 1134 | 5 |
| H3 | 0.00081 | 855 | 3601 | 3556 | 850 | 944 | 5 |
| H4 | 0.00227 | 305 | 1776 | 2142 | 2038 | 2264 | 5 |
| H5 | 0.00279 | 249 | 826 | 1217 | 17447 | 19386 | 5 |
| H6 | 0.00042 | 1665 | 3101 | 3050 | 2876 | 3196 | 0 |
| H7 | 0.00333 | 208 | 188 | 431 | 12205 | 13561 | 45 |
| H8 | 0.00292 | 237 | 322 | 610 | 14116 | 15684 | 45 |
| Average | 0.00179 | 638 | 2110 | 2236 | 6405 | 7116 | 14.4 |
| % CV | 64.8 | 78.6 | 71.4 | 59.2 | 108.7 | 108.7 | 132.0 |

TABLE 5
Individual and average compartmental parameters of MTR107
following IV administration to hemodialysis patients

| | k₁₀ | CV | k_dialysis | CV | k₁₂ | CV | k₂₁ | CV | V₁ | CV |
|---|---|---|---|---|---|---|---|---|---|---|
| Subject | 1/min | % | 1/min | % | 1/min | % | 1/min | % | mL/kg | % |
| H1 | 0.0013 | 65.5 | 0.0059 | 33.9 | 0.0509 | 20.8 | 0.0361 | 20.7 | 347 | 7.4 |
| H2 | 0.0000 | 0.0 | 0.0246 | 20.5 | 0.1460 | 11.3 | 0.0328 | 9.8 | 135 | 9.3 |
| H3 | 0.0000 | 0.0 | 0.0124 | 32.4 | 0.0767 | 21.4 | 0.0332 | 20.6 | 229 | 10.5 |
| H4 | 0.0000 | 0.0 | 0.0294 | 145.6 | 0.1048 | 29.2 | 0.0149 | 72.8 | 74 | 19.8 |
| Average | 0.0003 | 16.4 | 0.0181 | 58.1 | 0.0946 | 20.7 | 0.0293 | 31.0 | 196 | 11.7 |
| % CV | 200.0 | 200.0 | 59.8 | 100.9 | 43.1 | 35.5 | 33.2 | 91.6 | 60.5 | 47.1 |

The study was performed in hemodialysis patients at different general condition. The severity of renal disease of the patients was also variable. The concentration vs. time curves of subjects 3, 5, 6, 7, and 8 showed unreasonable concentrations at one/several individual time points that could be attributed to the effect of the hemodialysis procedure (possible interference of the uremic toxins in these patients with the selectivity of the analytical assay), or differences in sampling techniques at different time points. These fluctuations in the observed time course of the plasma concentrations hampered the results of the pharmacokinetic analysis, and precluded compartmental analysis in subjects 5-8.

The plasma concentration vs. time curves showed a rapid distributional phase that was completed 15-30 minutes after the intravenous administration, and afterwards the drug was slowly eliminated with first-order elimination kinetics (see FIG. 3). The individual data of the subjects 1-6 followed the same trend. The data of patients 7 and 8 showed a similar pattern of extreme fluctuations in plasma concentration around the average concentrations observed for subjects 1-6.

The results of non-compartmental analysis suggest that the sampling schedule applied in this study did not capture completely the time course of the plasma concentrations, and the % AUC that was extrapolated was more than 20% in 5 subjects (see Table 4). Therefore, the observed values of the major pharmacokinetic parameters may be significantly different from their true values.

Following intravenous administration of MTR107 to the hemodialysis patients the drug was cleared from the central circulation with mean elimination half-life (T½ β) of 638 min, and the mean MRT value was 869 min (see Table 4). The average total body clearance was 2.71 ml/min/kg, and the observed volume of distribution in the steady state was 2.1 L/kg.

The observed time course of MTR107 concentrations following intravenous (IV) administration to subjects 1-4 was successfully described by a modified two-compartment pharmacokinetic model (see Table 5 and FIG. 4). The individual data indicated that there was virtually no body clearance of MTR107 in 3 patients out of 4 (see Table 5). Based on the average results, the half-lives of the processes related to drug elimination and drug transfer between the compartments were 2310, 38.3, 73.3, and 23.7 min for k₁₀, k_{dialysis}, k₁₂, and k₂₁, respectively. The volume of the central compartment V₁ was 196 ml and was similar to the volume of extracellular fluid in humans (260 ml/kg).

### Example 3

### Simulation of the Concentration Vs. Time Curves of MTR107 in Hemodialysis Patients

### I. The simulations

The simulations of the multiple dosing of MTR107 to the hemodialysis patients were based on the applied pharmacokinetic model and the obtained values of the pharmacokinetic parameters (see FIG. 3 and Table 4).

Concentration vs. time data of subjects 5, 6, 7, and 8 were excluded from the analysis due to fluctuations in the obtained data that couldn't be attributed to the pharmacokinetic behavior of the drug, but rather to the differences in blood sampling procedure. Therefore, the modeling was based on the concentration vs. time data of subjects 1-4.
The simulations were performed for the following settings:

Multiple administration of the same dose of MTR107 at 0, 48, 96, 144, 192 and 240 hr (0, 2, 4, 6, 8, and 10 days).

The single dose of 0.3, 0.6, 0.9, 1.2 and 2.4 mg/kg.

The doses administered as a 3-min infusion.

The dialysis procedure was started 10 min before and was terminated 240 min after each administration of MTR107.

The kinetics of MTR107 clearance by the hemodialysis patients can't be determined precisely based on the results of pharmacokinetic study of MTR107 in hemodialysis patients due to the fact that the last blood sample was taken 720 min only after the drug administration. At that time point significant concentrations of MTR107 were detected, and the terminal slope of the decline in the drug concentrations could not be determined precisely. In addition, kinetics of MTR107 clearance by the hemodialysis patients could be subject to high inter-patient variability due to differences in renal functioning that is the major process responsible for the drug elimination from the body in healthy subjects.

Therefore, simulations of the concentration vs. time data were performed according to 2 scenarios that assumed presence or absence of MTR107 body clearance (k₁₀; resulting in presence or absence of elimination from the body at the time periods when the hemodialysis is not applied).

### II. The Simulation Results

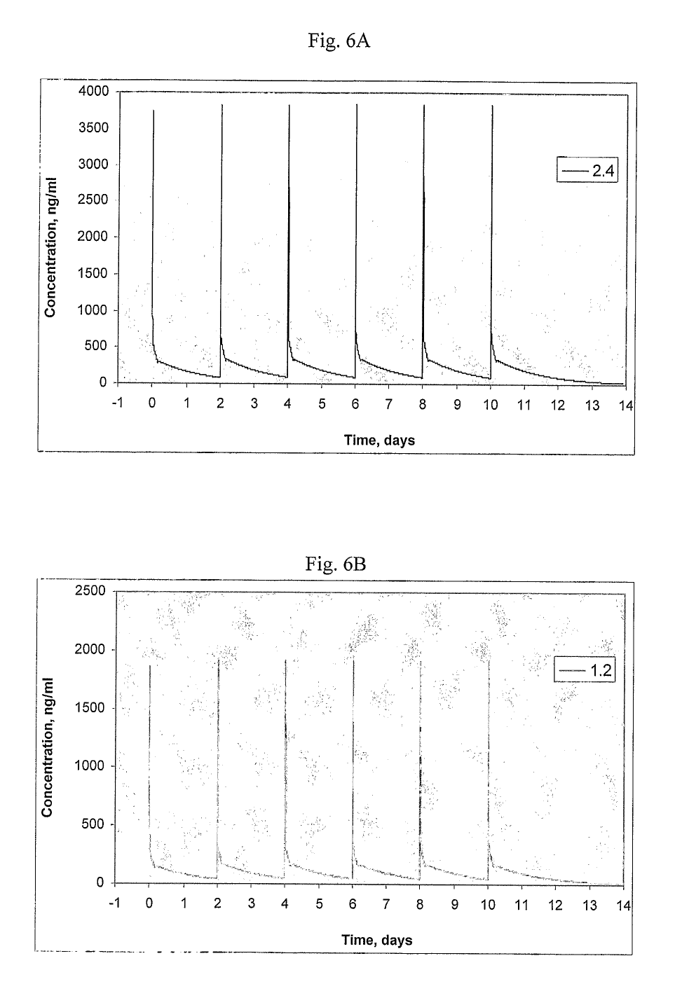

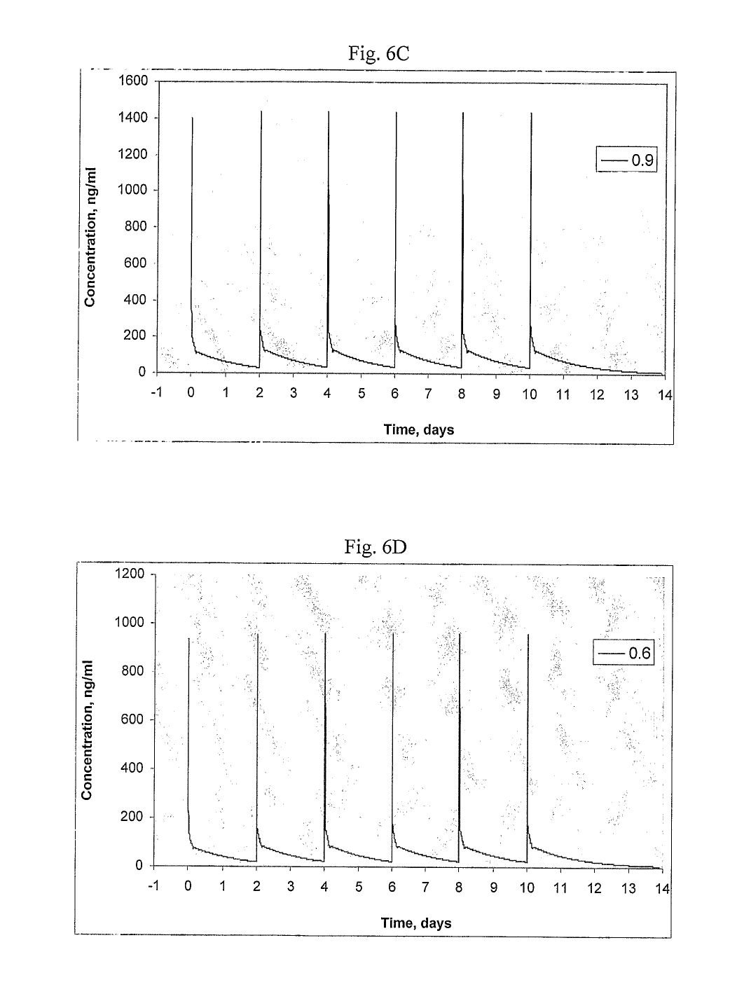

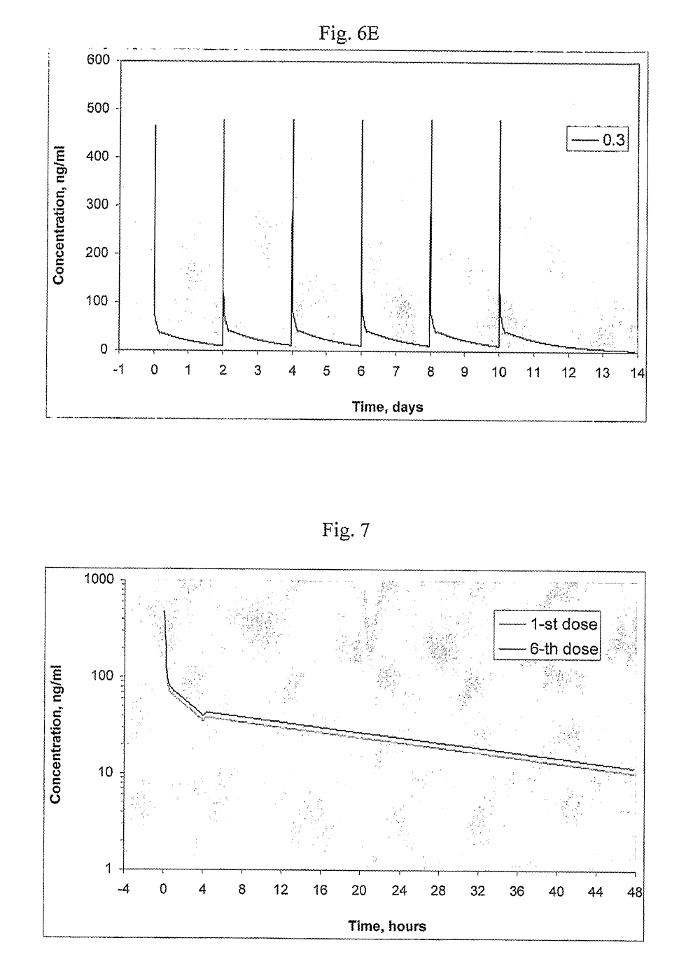

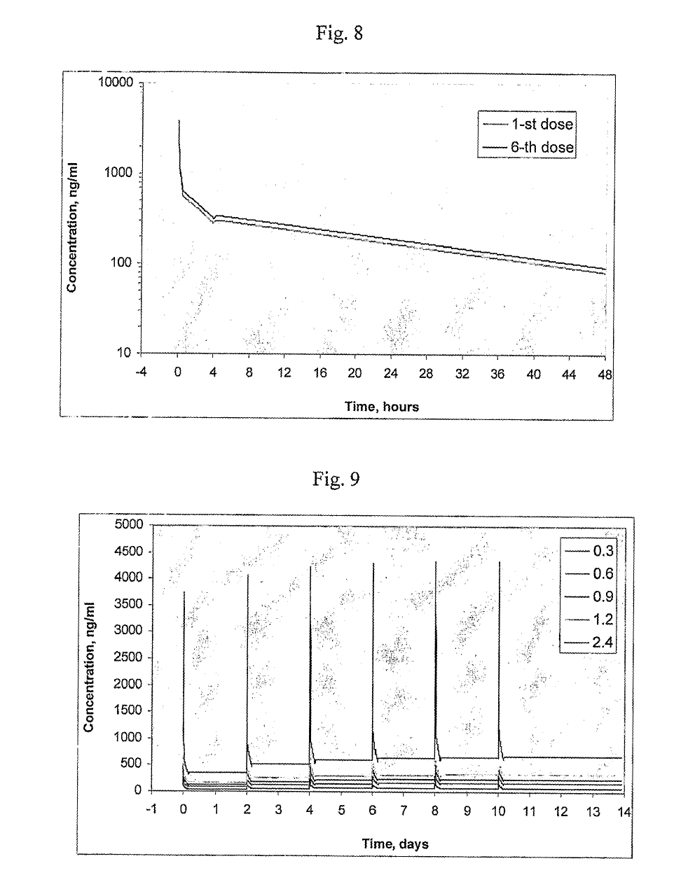

The results of Scenario 1: MTR107 elimination from the body (k₁₀) at the time periods when the hemodialysis is not applied, are presented in FIGS. 5-8.

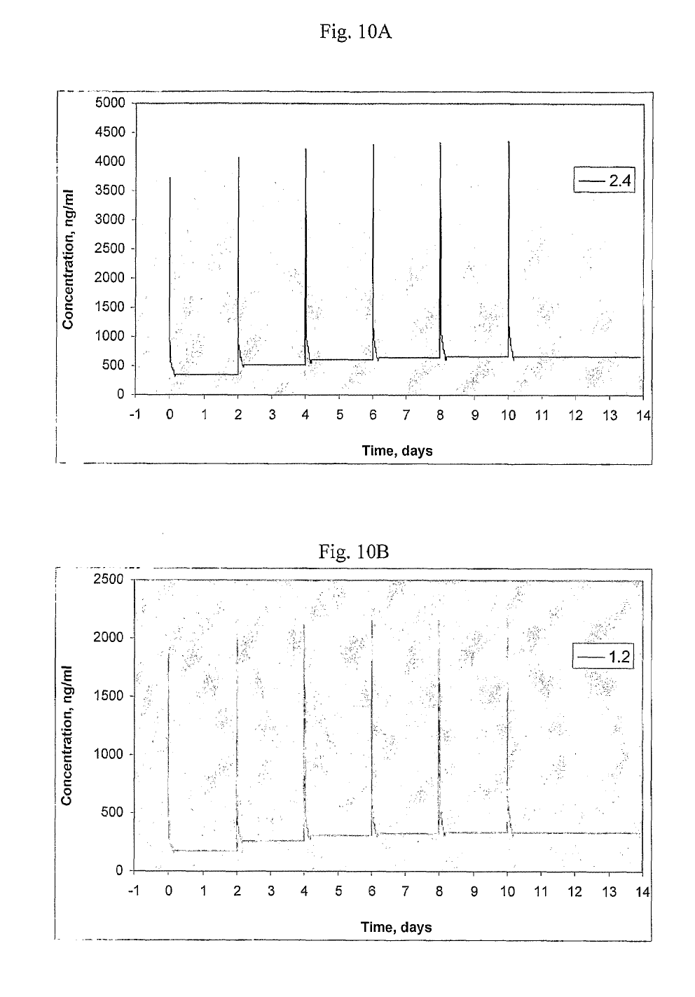

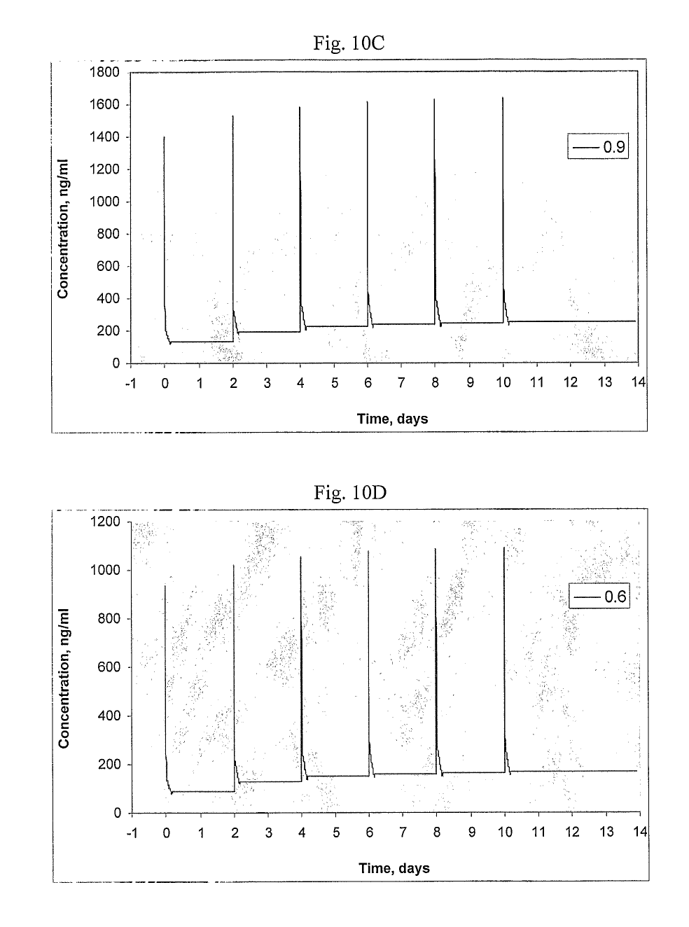

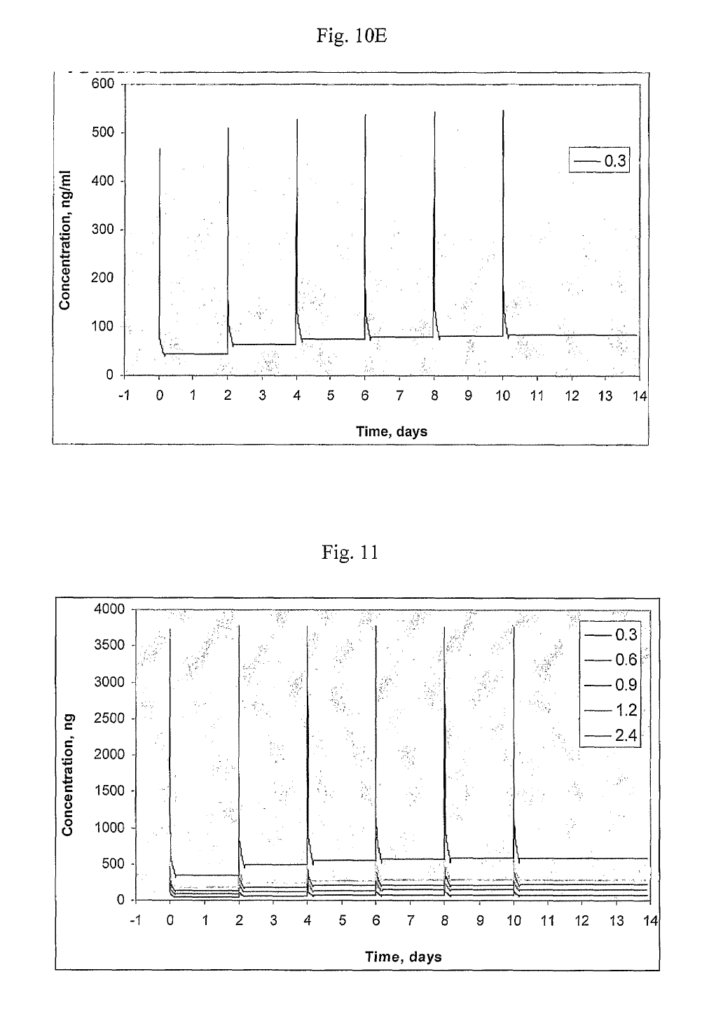

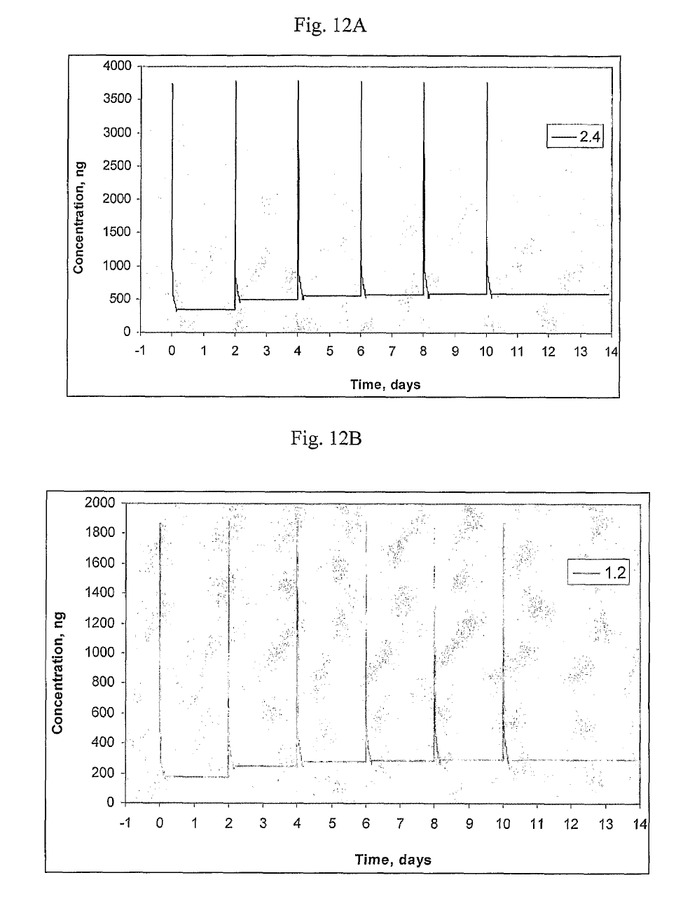

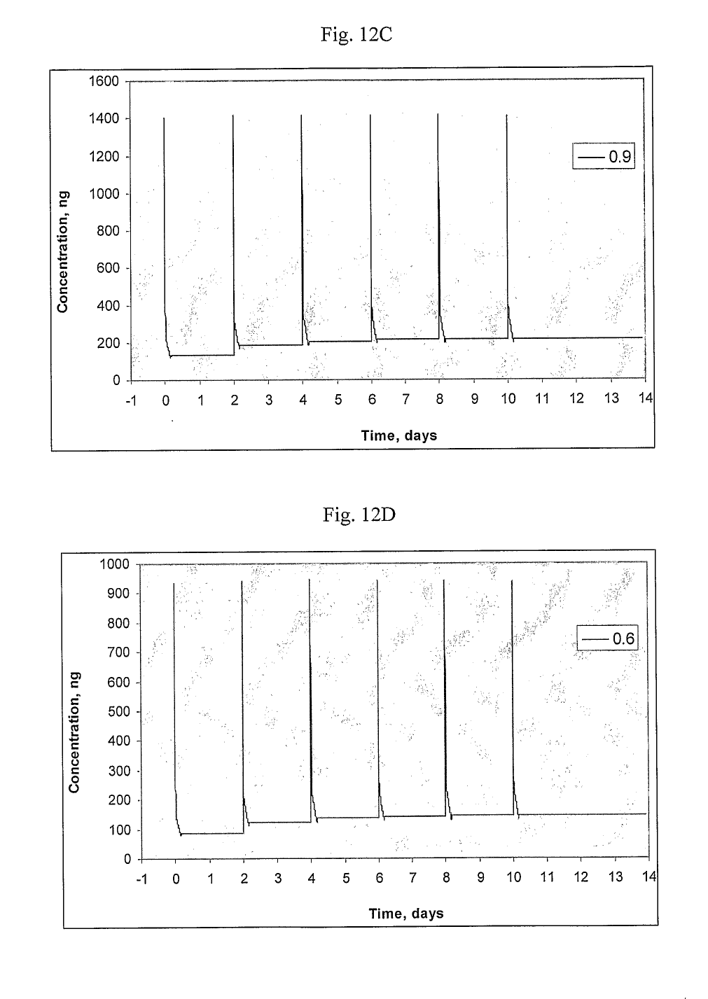

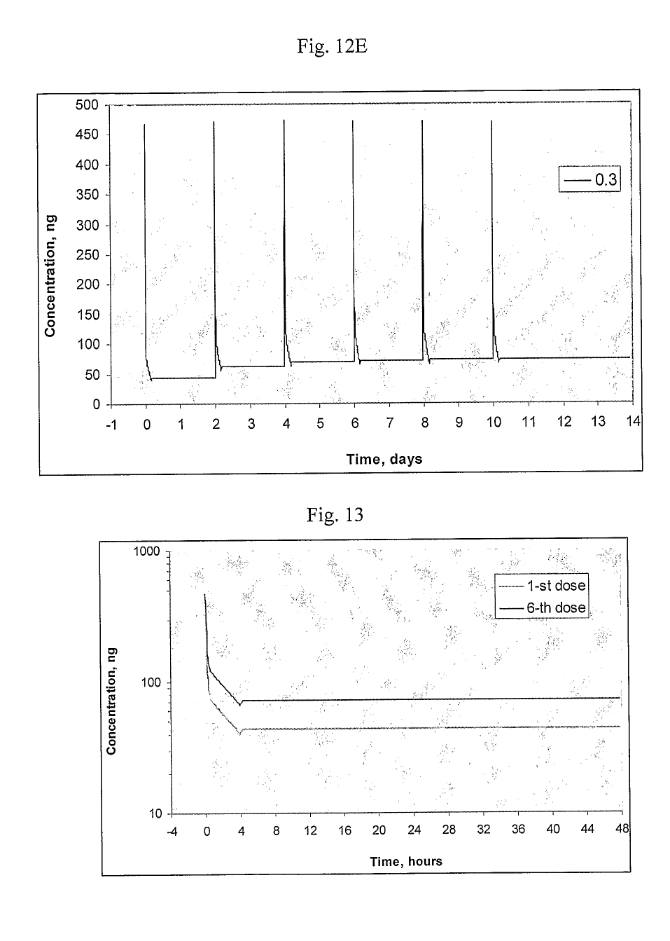

The results Scenario 2: absence of MTR107 elimination from the body at the time periods when the hemodialysis is not applied (k₁₀=0), are presented in FIGS. 9-13.

The results of the simulations indicate that multiple dosing of 0.3-2.4 mg/kg of MTR107 at 2-day intervals with concomitant hemodialysis is not expected to result in significant accumulation of the plasma drug concentrations if, despite major renal insufficiency, MTR107 is eliminated from the body in the absence of hemodialysis. In the case that MTR107 is eliminated from the body solely by the hemodialysis, significant accumulation of the plasma drug concentrations is expected to occur following multiple administration of 0.3-2.4 mg/kg doses.

The third part of the simulation includes modification of scenario 2: limited accumulation of MTR107 in the body and elimination from the body (k₁₀) at the time periods when the hemodialysis is not applied

In case that body clearance of MTR107 is negligible (k₁₀=0) in end stage renal disease, significant accumulation in drug concentrations is expected to occur and will result in significant increase in the peak and trough MTR107 plasma concentrations. The purpose of the last part of the simulation was to determine the multiple administration doses that would yield a minimal accumulation of the drug in the body.

The increase in trough concentrations of the drug could not be prevented for multiple administration dosage regimens because the administered dose could not be completely excreted during the 4-hr time period when hemodialysis is applied. On the other hand, increase in the peak concentrations of MTR107 could be prevented by sequential reduction of the drug dose.

Based on the Cₘₐₓ values obtained for scenario 2, the 1-6^{th} doses of MTR107 should be consequently decreased according to the following factors: 1.000, 0.9214, 0.8877, 0.8722, 0.8649, 0.8614 (e.g., for the multiple administration of 2.4 mg/kg, the 1-6^{th} doses should be 2.4, 2.211, 2.130, 2.093, 2.076, and 2.067 mg/kg, respectively). Results of the simulations according to this dosing scheme are presented in FIGS. 11-13.

Sequential reduction of the MTR107 dose during multiple dosing regimens was proposed to reduce the accumulation in the peak plasma levels of the drug in the case that MTR107 is eliminated from the body solely by the hemodialysis, and appropriate simulations were performed. While the current simulation approach focused on dose adjustments, an alternative option to reduce accumulation would be to increase the duration of the hemodialysis process.

### Example 4

### Pharmacokinetic and Pharmacodynamic Effects of MTR107 in ESRD Patients—A Phase II Clinical Trial Protocol

### Objectives

The objectives of the study were:
1. To characterize the pharmacokinetic profile of MTR107 administered preventively (at the beginning of dialysis sessions) in three escalating doses separated by washout periods.
2. To characterize the pharmacodynamic profile of subjects predisposed to develop hypotension during dialysis treated with MTR107 or with placebo.
3. To explore the pharmacokinetic model and the requirements for dose adjustment.
4. To collect data on exploratory efficacy end points.

### Overall Study Design

The study is a prospective, randomized, double blind, placebo controlled, dose range study analyzing the pharmacokinetic and pharmacodynamic profile of MTR107 in a population of patients predisposed to develop hypotension during dialysis. All patients enrolled have a documented history of predisposition to bouts of hypotension as defined by at least three events of hypotension per month during the last six months. Patients are randomly allocated to placebo or MTR107 treatment in each dose group prior to the beginning of the study. Ratio of drug to placebo treated patients is 3:1.

Treatment is started with the lowest dose as a single IV bolus administration. Both the drug and the placebo are administered as a slow IV bolus injection (10 ml of diluted medication or placebo injected over 3 minutes). Blood samples, exploratory parameters, adverse events, and vital signs are recorded continuously (with Holter) for the duration of the dialysis and one hour thereafter. During the washout period of 3 dialysis sessions, blood samples, exploratory parameters, adverse events, and vital signs are recorded only at the beginning and at the end of the dialysis.

### Sampling for Pharmacokinetic Data

For the pharmacokinetic analysis, 4 ml of blood are collected at baseline and at specific time points as described below. The study medication is injected 10 minutes after connecting the patient to the dialysis circuit. Blood samples are immediately centrifuged, and the plasma is separated and frozen at −20° C. Blood samples are also drawn from patients who are treated by placebo.

After study termination, randomization code is opened and MTR107 blood levels are analyzed only in patients administered the active study medication (MTR107). The drug levels in the blood are analyzed according to established and validated analytical methods.

### Pharmacodynamic Evaluations

Vital signs are continuously monitored, and recorded at specified time points, coinciding with blood sampling, during the course of the dialysis session, and up to 24 hours post administration. Similar data recording is done at beginning and end of dialysis sessions during the washout periods.

### Exploratory Parameters

The following exploratory parameters are collected throughout all dialysis sessions:

a. Number and type of medical interventions required for treatment of hypotension.

b. Presence of symptoms associated with intradialytic hypotension.

c. Efficiency of dialysis as reflected by Kt/V.

### Safety Assessment

Adverse events are recorded throughout the study period. Safety evaluation consists of monitoring hypertensive episodes, arrhythmias, incidence of adverse events, and deterioration in hepatic functions, and/or any other reported adverse event until the conclusion of the study.

### Inclusion Criteria

To be eligible for study entry patients must satisfy the following criteria:
1. Age 20-75 years, inclusive.
2. Presence of frequent bouts of hypotension defined as 3 or more intradialytic hypotensive events per month for the last six months prior to baseline, despite standard adjustments in dry weight.
3. ECG performed up to one month before study start.
4. Well-preserved hepatic function (within normal laboratory ranges).
5. Normal coagulation status at study entry as judged by PT-INR, PTT, fibrinogen and platelet count.
6. Willingness to participate and adhere to the study design.
7. Willingness to sign an informed consent form.

### Exclusion Criteria

Exclusion criteria includes:
1. Uncontrolled hypertension, >140/90 mmHg
2. Unstable angina.
3. Abnormal ECG which may indicate acute disease.
4. Current participation in another clinical trial involving an investigational drug/device, or participation in such a trial within the last 30 days.

### Timing Throughout the Study

### Overall Study Schedule

The total study duration is 6 months.

### Treatments

### Treatments Administered

Each dose or placebo is administered as a slow intravenous injection (10 ml of diluted medication or placebo over 3 minutes), 10 minutes after the beginning of the dialysis session.

### Study Medication (MTR107)

The study medication, MTR107, is administered starting first with the lowest dose of 0.3 mg/kg. Thereafter, at the fifth (5^{th}) and the ninth (9^{th} dialysis sessions, the dose is increased to 0.9 mg/kg, and 1.8 mg/kg, respectively.

### Placebo

Placebo is 10 ml of sterile saline solution for intravenous injection.

### Rescue Medication

In patients administered with placebo, or whenever blood pressure is not restored to acceptable levels as judged clinical by the physician, standard medical care is provided and recorded in the CRFs.

### Laboratory Testing

Laboratory tests including hematology, blood biochemistry, haemostatic parameters, markers of oxidative stress, will be performed at screening and at the end of treatment schedule (after completion of 9^{th} dialysis).

Efficiency of dialysis (Kt/V) is calculated before and after the completion of the dialysis prior to treatment, at each of the dialysis sessions when the patient is treated with MTR107, and at the last dialysis in the protocol.

### Identity of the Investigational Product

The cGMP research material is supplied by the sponsor in the form of single use, 2 ml sterile vials labeled with identification details, as well as with appropriate warning regarding its dedicated use in the study. The drug substance is S-ethylisothiouronium diethyl phosphate. The final drug product is a 10% (100 mg/ml) aqueous solution of S-ethylisothiouronium diethyl phosphate, which is to be kept at 4-8° C.

The drug is diluted at the site under sterile conditions, according to SOP provided by sponsor. The active drug is diluted with sterile saline solution for IV injection in a total volume of 10 ml that are injected over three minutes.

### Statistical Analysis

### Sample Size

The present study is a descriptive in nature and no formal hypothesis testing of a primary endpoint is intended. A power calculation is therefore inapplicable.

### Data Management

The data management system is SAS® version 8.2 with FSEDIT procedure (FSP and AF products).

The CRFs are collected from the site and are sent to Data Management by the Study Monitor. The CRFs are logged and the data are entered into the study database using double data entry with verification upon second entry. Text items/comments are entered once and checked manually against the CRFs. Queries are generated by programmed checks or entered manually. Once the queries are Quality Controlled, they are sent to the Monitor for resolution at the investigational site. Adverse events, concomitant diseases and concomitant therapies are coded according to coding dictionaries (COSTART, ICD-9 and WHO-ATC drug coding system).

### Statistical and Analytical Analysis

All statistical analysis are performed using SAS® version 8.2.

All safety analysis is based on the safety population, which include all randomized patients who receive study medication. Except where indicated, post-baseline missing data are not estimated. Complete individual patient listings by patient number and treatment group, if appropriate, are provided.

All key data are summarized in tables using appropriate summary statistics. Continuous endpoints are summarized as the, mean, minimum, maximum and standard deviation of n observations. Categorical endpoints are presented as frequency counts and percentages.

All adverse events and concomitant medications recorded during the study are coded using the COSTRAT and WHO-ATC drug coding system respectively.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It should be understood that the detailed description and specific examples, while indicating preferred embodiments of the invention, are given by way of illustration only, since various changes and modifications within the spirit and scope of the invention will become apparent to those skilled in the art from this detailed description.

1.-51. (canceled)

52. A method for preventing hypotension in a subject receiving hemodialysis, comprising administering to a subject a therapeutically effective amount of a compound having the general formula I: 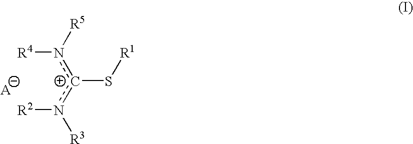
wherein:
R¹ is a linear or branched saturated or unsaturated alkylene, comprising one to eight carbon atoms optionally substituted with one or more substituent selected from the group consisting of halogen, primary, secondary, tertiary or quaternary amine, primary, secondary or tertiary alcohol, or interrupted by one or more heteroatom selected from the group consisting of O, N, and S;
R², R³, R⁴ and R⁵ are each independently a hydrogen, hydroxy, linear or branched lower alkyl, linear or branched lower alkenyl, linear or branched lower alkynyl, lower alkoxy, alkoxyalkyl, cycloalkyl, cycloalkylalkyl, lower thioalkoxy, nitro, amino, cyano, sulfonyl, haloalkyl, carboaryloxy, carboalkylaryloxy, alkyl sulfoxide, aryl sulfoxide, alkyl sulfone, aryl sulfone, alkyl sulfate, aryl sulfate, sulfonamide, thioalkyl, optionally substituted by halogen;
A⁻ is a physiologically acceptable anion;
and a pharmaceutically acceptable carrier or diluent.

53. The method of claim 52, wherein the physiologically acceptable anion is selected from the group consisting of an anion derived from a phosphorus containing acid, a phosphorous containing acid ester, a phosphorous containing acid amide, acetate, adipate, alginate, citrate, aspartate, benzoate, benzenesulfonate, bitartarate, bisulfate, butyrate, camphorate, camphorsulfonate, digluconate, glycerophosphate, hemisulfate, heptanoate, hexanoate, fumarate, 2-hydroxyethanesulfonate, isothionate, lactate, maleate, methanesulfonate, nicotinate, 2-naphthalenesulfonate, oxalate, palmoate, pectinate, 3-phenylpropionate, pivalate, propionate, succinate, tartrate, thiocyanate, phosphate, glutamate, bicarbonate, p-toluenesulfonate, chloride, bromide, iodide and undecanoate.

54. The method of claim 52, wherein the physiologically acceptable anion is a phosphorus containing acid.

55. The method of claim 54, wherein the phosphorus containing acid is selected from the group consisting of a mono-alkyl ester of a phosphorus containing acid and di-alkyl ester of a phosphorus containing acid.

56. The method of claim 52, wherein each of R², R³, R⁴ and R⁵ is hydrogen.

57. The method of claim 52, wherein R¹ is selected from the group consisting of a linear alkyl and branched alkyl.

58. The method of claim 57, wherein the compound is a S-alkylisothiouronium derivative having general formula (II): 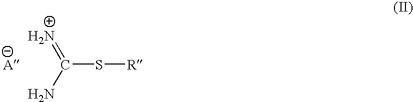
wherein:
R″ is a straight or branched alkyl, optionally substituted by halogen; and
A″⁽⁻⁾ is an anion derived from a phosphorous containing acid.

59. The method of claim 58, wherein the compound is selected from the group consisting of S-methylisothiouronium methylphosphite; S-methylisothiouronium dimethylphosphate; S-ethylisothiouronium metaphosphate; S-ethylisothiouronium ethylphosphite; S-ethylisothiouronium diethylphosphate; S-propylisothiouronium propylphosphite; S-isopropylisothiouronium metaphosphate; S-isopropylisothiouronium isopropylphosphite; S-butylisothiouronium dibutylphosphate; and S-isobutyl-isothiouronium isobutylphosphite.

60. The method of claim 52, wherein the compound is S-ethylisothiouronium diethylphosphate.

61. The method of claim 52, wherein the compound is formulated for injection.

62. The method of claim 52, wherein the compound is formulated for oral administration.

63. A method for stabilizing blood pressure during hemodialysis, comprising administering to a subject a therapeutically effective amount of a compound having the general formula I: 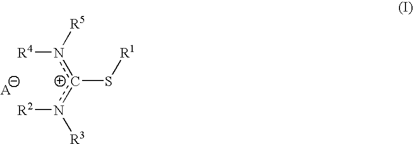
wherein,
R¹ is a linear or branched saturated or unsaturated alkylene, comprising one to eight carbon atoms optionally substituted with one or more substituent selected from the group consisting of halogen, primary, secondary, tertiary or quaternary amine, primary, secondary or tertiary alcohol, or interrupted by one or more heteroatom selected from the group consisting of O, N, and S;
R², R³, R⁴ and R⁵ are each independently a hydrogen, hydroxy, linear or branched lower alkyl, linear or branched lower alkenyl, linear or branched lower alkynyl, lower alkoxy, alkoxyalkyl, cycloalkyl, cycloalkylalkyl, lower thioalkoxy, nitro, amino, cyano, sulfonyl, haloalkyl, carboaryloxy, carboalkylaryloxy, alkyl sulfoxide, aryl sulfoxide, alkyl sulfone, aryl sulfone, alkyl sulfate, aryl sulfate, sulfonamide, thioalkyl, optionally substituted by halogen;
A⁻ is a physiologically acceptable anion;
and a pharmaceutically acceptable carrier or diluent.

64. The method of claim 63, wherein the physiologically acceptable anion is selected from the group consisting of an anion derived from a phosphorus containing acid, a phosphorous containing acid ester, a phosphorous containing acid amide, acetate, adipate, alginate, citrate, aspartate, benzoate, benzenesulfonate, bitartarate, bisulfate, butyrate, camphorate, camphorsulfonate, digluconate, glycerophosphate, hemisulfate, heptanoate, hexanoate, fumarate, 2-hydroxyethanesulfonate, isothionate, lactate, maleate, methanesulfonate, nicotinate, 2-naphthalenesulfonate, oxalate, palmoate, pectinate, 3-phenylpropionate, pivalate, propionate, succinate, tartrate, thiocyanate, phosphate, glutamate, bicarbonate, p-toluenesulfonate, chloride, bromide, iodide and undecanoate.

65. The method of claim 63, wherein the physiologically acceptable anion is a phosphorus containing acid.

66. The method of claim 65, wherein the phosphorus containing acid is selected from the group consisting of a mono-alkyl ester of a phosphorus containing acid and di-alkyl ester of a phosphorus containing acid.

67. The method of claim 63, wherein each of R², R³, R⁴ and R⁵ are hydrogen.

68. The method of claim 63, wherein R¹ is selected from the group consisting of a linear alkyl and branched alkyl.

69. The method of claim 68, wherein the compound is a S-alkylisothiouronium derivative having general formula (II): 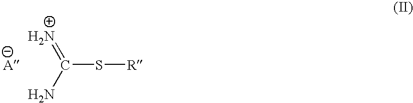
wherein
R″ is a straight or branched alkyl, optionally substituted by halogen; and
A″⁽⁻⁾ is an anion derived from a phosphorous containing acid.

70. The method of claim 63, wherein the compound is selected from the group consisting of S-methylisothiouronium methylphosphite; S-methylisothiouronium dimethylphosphate; S-ethylisothiouronium metaphosphate; S-ethylisothiouronium ethylphosphite; S-ethylisothiouronium diethylphosphate; S-propylisothiouronium propylphosphite; S-isopropylisothiouronium metaphosphate; S-isopropylisothiouronium isopropylphosphite; S-butylisothiouronium dibutylphosphate; and S-isobutyl-isothiouronium isobutylphosphite.

71. The method of claim 63, wherein the compound is S-ethylisothiouronium diethylphosphate.

72. The method of claim 63, wherein the compound is formulated for injection.

73. The method of claim 63, wherein the compound is formulated for oral administration.
